(19) United States
(12) Patent Application Publication
(10) Pub. No.: US 2005/0283179 A1
(43) Pub. Date: Dec. 22, 2005

(54) Introducer sheath

(76) Inventor: David J. Lentz, La Jolla, CA (US)

Correspondence Address: NEIL K. NYDEGGER;NYDEGGER & ASSOCIATES, 348 Olive Street, San Diego, CA, 92103, US

(21) Appl. No.: 10/869,996

(22) Filed: Jun. 17, 2004

(51) Int. Cl. A61M 29/00

(52) U.S. Cl. 606/192; 264/145; 607/117

(57) ABSTRACT

An introducer sheath includes an elongated hollow hypotube that is formed with a spiral cut that extends between the ends of the hypotube and extends through the hypotube from its outer surface to its inner surface. During manufacture, the spiral cut is made with a pitch angle (α) that can be varied along the length of the hypotube to provide different sections of the hypotube with different degrees of flexibility. A polymer coating is positioned on the outer surface of the hypotube to establish a lubricious coating for the introducer sheath.

### FIELD OF THE INVENTION

The present invention pertains generally to medical devices that are to be advanced into the vasculature of a patient. More particularly, the present invention pertains to introducer sheaths. The present invention is particularly, but not exclusively, useful for introducer sheaths having variable rigidity with increasing flexibility in the distal direction.

### BACKGROUND OF THE INVENTION

Introducer sheaths, as the name suggests, are medical devices that are used to establish extracorporeal access into the vasculature of a patient. More specifically, introducer sheaths are used to effectively establish an access port into the vasculature through which other interventional medical devices can be subsequently introduced and withdrawn. Typically, during a particular medical procedure, several different types of medical devices may need to be inserted into and withdrawn from the vasculature through the same introducer sheath.

In order for an introducer sheath to effectively perform its intended purpose, it needs to exhibit several specific structural and functional capabilities. For one, the introducer sheath must provide a passageway of sufficient size to accommodate the insertion and withdrawal of medical devices through the passageway. Also, for ease of use, the preference here is for a sheath wherein the percentage of cross sectional area available for use as a passageway is maximized. On the other hand, the overall outside diameter of the sheath must not be too large.

With the above in mind, the amount of material that can be used for constructing the structure of the sheath should be minimal. This raises a competing concern in that, on the one hand, the structure of the sheath surrounding its passageway needs to be small. On the other hand, it must also have sufficient strength to maintain the integrity of the device during its use. Specifically, the sheath must be capable of resisting collapse and withstanding any tendency to kink. Further, it is necessary for the introducer sheath to have sufficient flexibility to allow it to be properly advanced and positioned in the vasculature. Ideally, the introducer sheath will also have variable flexibility along its length, with the most flexibility being at its distal end.

Insofar as strength capabilities for an introducer sheath are concerned, the incorporation of a tube-like structure that is made of a high strength material, such as stainless steel, is preferable. Specifically, a hypotube of a type well known in the pertinent art is quite suitable for this purpose. Hypotubes, however, are very stiff and inflexible.

In light of the above, it is an object of the present invention to provide an introducer sheath that has the “hoop strength” needed to resist a collapsing or kinking of the sheath during its use. Another object of the present invention is to provide an introducer sheath that has a variable flexibility along its length for effectively positioning the sheath into the vasculature of a patient. Still another object of the present invention is to provide an introducer sheath that is relatively simple to manufacture, is easy to use and is comparatively cost effective.

### SUMMARY OF THE INVENTION

An introducer sheath in accordance with the present invention includes an elongated hollow hypotube which is coated with a flexible polymer material. In particular, the hypotube is formed with a spiral cut that extends the length of the tube from its proximal end to its distal end. Importantly, the spiral cut also extends completely through the hypotube from its outer surface to its inner surface. With this structure, the spiral cut can be characterized at any axial location along the hypotube by a pitch angle (α). Specifically, the pitch angle (α) is measured between the axis of the hypotube and the inclination of the spiral cut at a location. Functionally, the pitch angle (α) is indicative of the flexibility of the hypotube at each particular location.

As an alternative to the above description, the introducer sheath of the present invention can be described as being a ribbon-like band that is configured as a helical spiral. In this description, the spiral band is positioned around an axis, at a predetermined radial distance from the axis. One edge of the band is juxtaposed at an extremely short axial distance from its other edge to form a flexible tube. As a practical matter, this axial distance is determined by the gap width of the spiral cut mentioned above in the first description of the introducer sheath.

In line with either of the descriptions given above for the introducer sheath, the result is a tube (hypotube) that has a proximal end and a distal end with a lumen extending through the tube from end to end. Also, in each case, a flexible polymer material is positioned on the outer surface of the band as a coating that will provide a substantially fluid-tight condition for the lumen and give the introducer sheath a lubricious quality that will facilitate its advancement into the vasculature of a patient.

As an additional feature for the present invention, the polymer coating on the hypotube may be formed with an inflation lumen that extends from the proximal end of the hypotube to substantially the distal end thereof. Further, a balloon can be mounted on the hypotube at its distal end and connected in fluid communication with the inflation lumen. Still further, a fluid pump may be connected in fluid communication with the inflation lumen at the proximal end of the hypotube for selectively inflating the balloon.

Insofar as the spiral cut is concerned, it is an important aspect of the present invention that the pitch angle (α) can be varied along the length of the introducer sheath. Preferably, variations in the pitch angle (α) will be in a range between forty-five and ninety degrees. With this in mind, the introducer sheath may be characterized as having a plurality of sections, wherein the pitch angle (α) has a respective average value for each of the different sections. For example, for a sheath having three sections, there may be a first pitch angle (α₁) having an average value equal to approximately seventy-five degrees in a first section, a second pitch angle (α₂) having an average value equal to approximately eighty degrees in a second section, and a third pitch angle (α₃) having an average value equal to approximately eighty-five degrees in a third section. In this example, the introducer sheath would exhibit greater flexibility wherever the pitch angle is greater.

In the manufacture of the introducer sheath, the spiral cut is preferably accomplished using an industrial laser cutting system. Also, the hypotube is preferably made of either stainless steel or NITINOL. Further, the flexible polymer coating is preferably a PEBAX type material.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1A is a perspective view of an introducer sheath in a straight configuration in accordance with the present invention;

FIG. 1B is a perspective view of the introducer sheath shown in FIG. 1A in a curved configuration;

FIG. 2 is a cross-sectional view of the introducer sheath as seen along the line 2-2 in FIG. 1A;

FIG. 3A is a cross-sectional view of the introducer sheath as seen along the line 3A-3A in FIG. 1B;

FIG. 3B is a cross-sectional view of the introducer sheath as seen along the line 3B-3B in FIG. 1B; and

FIG. 3C is a cross-sectional view of the introducer sheath as seen along the line 3C-3C in FIG. 1B.

### DESCRIPTION OF THE PREFERRED EMBODIMENTS

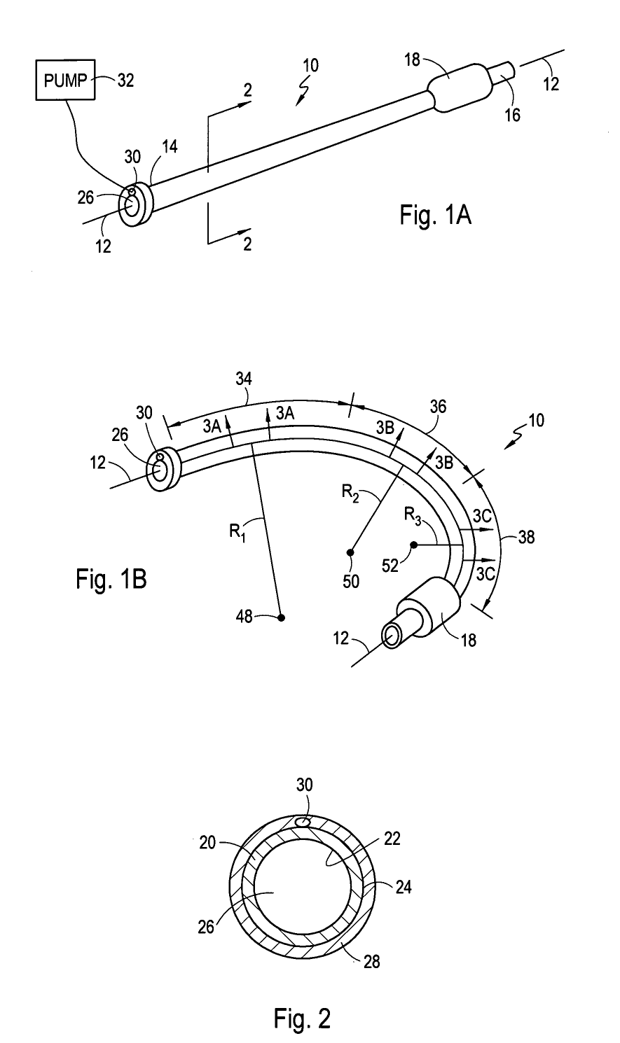

Referring initially to FIGS. 1A and 1B, an introducer sheath in accordance with the present invention is shown and is generally designated 10. As shown, the introducer sheath 10 is an elongated structure defining an axis 12 and having a proximal end 14 and a distal end 16. Also, the introducer sheath 10 may include an inflatable balloon 18 located at, or near, the distal end 16 of the sheath 10. As intended for the present invention, the introducer sheath 10 is to be inserted into the vasculature of a patient (not shown) to establish an access port for the subsequent insertion and withdrawal of other medical devices (also not shown) into and out of the patient.

The construction of the introducer sheath 10 will, perhaps, be best appreciated with reference to FIG. 2. There it will be seen that the introducer sheath 10 includes a hypotube 20 that has an inner surface 22 and an outer surface 24. More specifically, the inner surface 22 surrounds a central lumen 26 of the sheath 10, and a flexible polymer coating 28 is positioned on the outer surface 24 of the hypotube 20. Further, as shown in FIG. 2, the coating 28 can be formed with an inflation lumen 30 that extends between the ends 14 and 16 of hypotube 20 to connect a fluid pump 32 in fluid communication with the balloon 18 (see FIG. 1A). Thus, if incorporated, the balloon 18 can be inflated by the fluid pump 32 to anchor the introducer sheath 10 in the vasculature during its use. For purposes of the present invention, the hypotube 20 is preferably made of a metallic material such as stainless steel or NITINOL, and the coating 28 is preferably made of a flexible polymer such as the material commercially known as PEBAX.

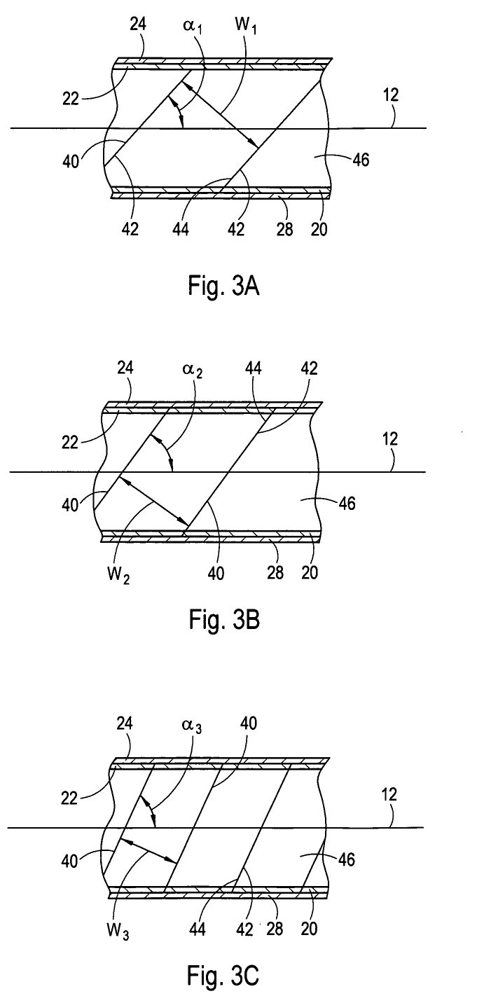

As will be appreciated by cross-referencing FIG. 1A with FIG. 1B, the introducer sheath 10 is flexible. More specifically, as envisioned for the present invention, the introducer sheath 10 can be manufactured to have a variable flexibility along its length between the ends 14 and 16. To disclose this attribute of the present invention, the introducer sheath 10 is best considered as having several sequential sections. With this in mind, the sections 34, 36 and 38 shown in FIG. 1B are only exemplary. The variable flexibility of the introducer sheath 10 can then be best discussed by comparing the relative structural aspects of the sections 34, 36 and 38. Portions of these sections 34, 36 and 38 are respectively shown in FIG. 3A, FIG. 3B and FIG. 3C.

Referring to the FIGS. 3A, 3B and 3C, it is to be appreciated that the hypotube 20 is formed With a spiral cut 40 that extends along the length of the hypotube 20 from its proximal end 14 to its distal end 16. Further, the spiral cut 40 extends through the hypotube 20 between its inner surface 22 and its outer surface 24. In more detail, the spiral cut 40 creates a gap between an edge 42 and an edge 44 that essentially forms the hypotube 20 as a helical shaped band 46 having a width “w” between the edges 42 and 44. Further, the inclination of the spiral cut 40 (gap) from the axis 12 through a pitch angle (α) is indicative of the flexibility of the hypotube 20 at the location where the pitch angle (α) is measured. More specifically, the pitch angle (α) is measured between the axis 12 and a projection of the spiral cut 40 onto a plane containing the axis 12. Thus, the increase in pitch angle (α) from α₁ (FIG. 3A), to α₂ (FIG. 3B), and eventually to α₃ (FIG. 3C) is indicative of an increasing flexibility for the hypotube 20 in the distal direction from end 14 to end 16. It is also to be appreciated by comparing FIGS. 3A, 3B and 3C, that this increase in flexibility is also indicated by a diminution in the width “w” of the band 46. Specifically, with the increase in flexibility, the width “w” decreases from w₁ (FIG. 3A), to w₂ (FIG. 3B), and eventually to w₃ (FIG. 3C).

Preferably, the spiral cut 40 is made using well known industrial laser cutting techniques. The spiral cut 40, however, may be made by several other means well known in the pertinent art. In any event, during manufacture, the pitch angle (α) and the width “w” can be established at locations in sections along the length of the hypotube 20 (e.g. sections 34, 36 and 38) to give it the desired flexibility. For example, the pitch angle (α) may be varied within a range between forty-five and ninety degrees and have a respective average value for each of the sections 34, 36 and 38. Again, by way of example, the pitch angle (α₁) in section 34 may have an average value equal to approximately seventy-five degrees while pitch angle (α₂) in section 36 has an average value equal to approximately eighty degrees in a second section. Pitch (α₃) in section 38 may then have an average value equal to approximately eighty-five degrees in a third section. The hypotube 20 can then be covered with the polymer coating 28 to provide a fluid-tight condition for the central lumen 26 and give the introducer sheath 10 a lubricious quality that will facilitate insertion of the sheath 10 into the vasculature of a patient.

The consequence of the present invention is an introducer sheath 10 that, due to the structural qualities of the hypotube 20, has sufficient strength to resist collapse and avoid kinking. At the same time, due to the geometry of the spiral cut 40, the sheath 10 has the desired degrees of variable flexibility. In combination, this provides an introducer sheath 10 having the different bending characteristics that are substantially shown in FIG. 1B. Specifically, section 34 is generally characterized by bending about a point 48 with a radius of curvature R₁. Similarly, section 36 is generally characterized by bending about a point 50 with a radius of curvature R₂, and section 38 is generally characterized by bending about a point 52 with a radius of curvature R₃. In this case, as shown in FIG. 1B, R₁ is greater than R₂, and R₂ is greater than R₃ (R₁>R₂>R₃).

While the particular Introducer Sheath as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

1. An introducer sheath which comprises:
an elongated hollow hypotube defining an axis and having a proximal end and a distal end with an inner surface and an outer surface therebetween, wherein the hypotube is formed with a spiral cut extending from the proximal end to the distal end and through the hypotube from the outer surface to the inner surface, and further wherein the spiral cut is characterized at an axial location on the hypotube by a pitch angle (α); and
a flexible polymer material positioned on the outer surface of the hypotube to establish a lubricious coating for the introducer sheath.

2. An introducer sheath as recited in claim 1 wherein the coating on the hypotube is formed with an inflation lumen extending from the proximal end of the hypotube to substantially the distal end thereof and wherein the introducer sheath further comprises:
a balloon mounted on the hypotube at the distal end thereof, with the balloon connected in fluid communication with the inflation lumen; and
a fluid pump connected in fluid communication with the inflation lumen at the proximal end of the hypotube for selectively inflating the balloon.

3. An introducer sheath as recited in claim 1 wherein the pitch angle (α) is measured from the axis to an inclination of the spiral cut at each location.

4. An introducer sheath as recited in claim 3 wherein the pitch angle (α) is in a range between forty-five and ninety degrees.

5. An introducer sheath as recited in claim 3 wherein the introducer sheath is characterized by a plurality of sections and wherein the pitch angle (α) has a respective average value for each section.

6. An introducer sheath as recited in claim 5 wherein the sheath has three sections, with a first pitch angle (α₁) having an average value equal to approximately seventy-five degrees in a first section, a second pitch angle (α₂) having an average value equal to approximately eighty degrees in a second section, and a third pitch angle (α₃) having an average value equal to approximately eighty-five degrees in a third section.

7. An introducer sheath as recited in claim 3 wherein the pitch angle (α) establishes a radius of curvature (R) for the introducer sheath at the location.

8. An introducer sheath as recited in claim 1 wherein the hypotube is made of stainless steel.

9. An introducer sheath as recited in claim 1 wherein the polymer is PEBAX.

10. An introducer sheath which comprises:
a ribbon-like band having a first edge and a second edge with a width therebetween, wherein the band is configured as a spiral around an axis with the band positioned at a predetermined radial distance from the axis and the first edge is juxtaposed at a predetermined axial distance from the second edge to form a flexible tube having a first end and a second end with a lumen therebetween; and
a flexible polymer material positioned on the band to establish a substantially fluid-tight condition for the lumen.

11. An introducer sheath as recited in claim 10 wherein the first end is a proximal end and the second end is a distal end, wherein the polymer material establishes a coating on the tube, and wherein the coating is formed with an inflation lumen extending from the proximal end of the tube to substantially the distal end thereof and wherein the introducer sheath further comprises:
a balloon mounted on the tube at the distal end thereof, with the balloon connected in fluid communication with the inflation lumen; and
a fluid pump connected in fluid communication with the inflation lumen at the proximal end of the tube for selectively inflating the balloon.

12. An introducer sheath as recited in claim 11 wherein, for a substantially constant predetermined axial distance and a substantially constant predetermined radial distance, the width of the band is selectively varied between the proximal end and the distal end of the tube.

13. An introducer sheath as recited in claim 12 wherein the introducer sheath is characterized by a plurality of sections and wherein the width of the band has a respective average value for each section.

14. An introducer sheath as recited in claim 13 wherein the sheath has three sections, with a first width (w₁) in a first section, a second width (w₂) in a second section, and a third width (W₃) in a third section, and wherein the first width is greater than the second width and the second width is greater than the third width (w₁>W₂>W₃).

15. An introducer sheath as recited in claim 14 wherein the width of the band establishes a characteristic radius of curvature (R) for each respective section of the introducer sheath.

16. An introducer sheath as recited in claim 10 wherein the tube is made of stainless steel.

17. An introducer sheath as recited in claim 10 wherein the polymer material is PEBAX.

18. A method for manufacturing an introducer sheath which comprises the steps of:
providing an elongated hollow hypotube defining an axis and having a proximal end and a distal end with an inner surface and an outer surface therebetween;
forming a spiral cut extending from the proximal end of the hypotube to the distal end thereof and through the hypotube from the outer surface to the inner surface with the spiral cut being characterized at an axial location on the hypotube by a pitch angle (α) measured from the axis to an inclination of the spiral cut; and
positioning a flexible polymer material on the outer surface of the hypotube to establish a lubricious coating for the introducer sheath.

19. A method as recited in claim 18 further comprising the steps of:
creating an inflation lumen in the coating with the inflation lumen extending from the proximal end of the hypotube to substantially the distal end thereof;
mounting a balloon on the hypotube at the distal end thereof, with the balloon connected in fluid communication with the inflation lumen; and
connecting a fluid pump in fluid communication with the inflation lumen at the proximal end of the hypotube for selectively inflating the balloon.

20. A method as recited in claim 18 further comprising the steps of:
establishing at least three sections for the introducer sheath wherein each section has a pitch angle (α) with a respective average value; and
selectively varying the pitch angle (α) with a first pitch angle (α₁) having an average value equal to approximately seventy-five degrees in a first section, a second pitch angle (α₂) having an average value equal to approximately eighty degrees in a second section, and a third pitch angle (α₃) having an average value equal to approximately eighty-five degrees in a third section.
